(19) United States
(12) Patent Application Publication
(10) Pub. No.: US 2009/0215729 A1
(43) Pub. Date: Aug. 27, 2009

(54) MICROPARTICLE COMPOSITIONS TO MODIFY CANCER PROMOTING CELLS

(76) Inventors: Erin M. Johnson, Napa, CA (US); Mark E. Johnson, Napa, CA (US)

Correspondence Address: FENWICK & WEST LLP, SILICON VALLEY CENTER, 801 CALIFORNIA STREET, MOUNTAIN VIEW, CA, 94041, US

(21) Appl. No.: 12/389,325

(22) Filed: Feb. 19, 2009

Related U.S. Application Data
(60) Provisional application No. 61/066,364, filed on Feb. 19, 2008.
(60) Provisional application No. 61/066,361, filed on Feb. 19, 2008.

(51) Int. Cl. A61K 31/663; A61P 35/00

(52) U.S. Cl. 514/108

(57) ABSTRACT

This invention provides pharmaceutical compositions and methods related to the prevention and treatment of primary tumors and metastatic, malignant or spreading cancers by selectively targeting cancer associated myeloid derived cells by the targeted delivery of a bisphosphonate formulated with a non-liposomal particle carrier. In some aspects, the bisphosphonate particles have one or more properties suitable for phagocytosis by cancer associated myeloid derived cells and release of the bisphosphonate within the macrophages. Advantageously, administering the particles to a subject reduces the level and/or activity of cancer associated myeloid derived cells in the subject.

### CROSS REFERENCE TO RELATED APPLICATIONS

This application claims the benefit of U.S. Provisional Application No. 61/066,364, filed Feb. 19, 2008, and U.S. Provisional Application No. 61/066,361, filed Feb. 19, 2008, both of which are herein incorporated by reference in their entirety.

### FIELD

Provided herein are methods and compositions for treating or preventing the growth, invasion and/or metastasis of a tumor by administering a composition comprising a bisphosphonate associated with a non-liposomal particulate carrier. The non-liposomal bisphosphonate particles advantageously target cancer-associated macrophages. Also provided herein are pharmaceutical compositions useful in treating and preventing cancer and tumor growth, invasion and/or metastases, comprising a bisphosphonate and a non-liposomal particulate carrier.

### BACKGROUND

Bisphosphonates are molecules characterized by two C—P bonds. If the two bonds are located on the same carbon atom (P—C—P) they are termed germinal bisphosphonates. The bisphosphonates have a chemical structure similar to that of inorganic pyrophosphate, an endogenous regulator of bone mineralization. While inorganic pyrophosphate is comprised of two phosphate groups linked by a phosphoanhydride bond, bisphosphonates are comprised of two phosphonate groups linked by phosphoether bonds to a central carbon atom. Unlike the pyrophosphate bond, the bisphosphonate bond is highly resistant to hydrolysis under acidic conditions or enzymatic action. Two additional covalent bonds to the central carbon in the bisphosphonates can be formed with carbon, oxygen, halogen, sulfur or nitrogen atoms, giving rise to a variety of possible structures. Like pyrophosphate, the two phosphate groups on the bisphosphonates readily form complexes with divalent metal ions such as Ca, Mg and Fe in a bidentate or tridentate manner.

Bisphosphonates have been clinically used mainly as (a) antiosteolytic agents in patients with increased bone destruction, especially Paget's disease, tumor bone disease and osteoporosis; (b) skeletal markers for diagnostic purposes; (c) inhibitors of calcification in patients with ectopic calcification and ossification, and (d) antitartar agents added to toothpaste (Fleisch, H., 1997, in: Bisphosphonates in bone disease. Parthenon Publishing Group Inc., 184-184). Furthermore, being highly hydrophilic and negatively charged, bisphosphonates in their free form are almost incapable of crossing cellular membranes.

The complexation of bisphosphonates to Ca ions is the basis of the bone-targeting property of these compounds. Bisphosphonates have therefore been widely used for treating osteolytic bone disease and osteoporosis, and to inhibit development of bone metastases or excessive bone resorption. It has also been observed that patients with bone metastasis, rheumatoid arthritis and osteoarthritis experience decreased pain following treatment with bisphosphonates (e.g., US patent application 20040176327).

U.S. Pat. Nos. 6,984,400 and 6,719,998 disclose methods for treating restenosis by administering nanoparticle formulation of certain bisphosphonates, which were taken up by macrophages implicated in the progression of restenosis and found to deplete such macrophages. US patent application 20060210639 describes bisphosphonate particles used for treating and/or preventing various bone disorders, including osteoporosis, which can include post-menopausal osteoporosis, steroid-induced osteoporosis, male osteoporosis, disease-induced osteoporosis, idiopathic osteoporosis; Paget's disease; abnormally increased bone turnover; periodontal disease; localized bone loss associated with periprosthetic osteolysis; and bone fractures.

Studies have reported the use of bisphosphonates to treat tumor growth and/or metastasis. For example, US patent application 20040176327 discloses methods of treating angiogenesis by administering a bisphosphonate to a patient who may be suffering from tumor growth or metastasis. The bisphosphonates are described as having an inhibitory effect on the angiogenic growth of endothelial capillaries associated with tumor growth and invasion. Similarly, WO 99/29345 describes methods of inhibiting tumor growth by administering a compound that reduces the level of activated macrophages in the region of a tumor. In one aspect, the methods involve administering a compound that is selectively cytotoxic for activated macrophages, such as a bisphosphonate (referred to as a “disphosphonate”). Neither US patent application 20040176327 nor WO 99/29345 describe the use of particulate bisphosphonate formulations.

Liposomal clodronate is known as a potent anti-macrophage agent, both in vitro and in vivo (van Rooijen N, et al., Cell Tissue Res. 238:355-358 [1984]; Seiler P et al., Eur. J. Immunol. 27:2626-2633 [1997]; van Rooijen N et al., J. Immunol. Meth. 193:93-99 [1996]; van Rooijen N et al., J. Immunol. Meth. 174:83-93 [1994]; van Rooijen, N., J. Immol. Meth. 124(1):1-6 [1989]), and recent studies have reported the use of liposomal clodronate to deplete tumor-associated macrophages (US patent application 20070218116 and Zeisberger et al., Brit. J. Cancer 95:272-281 [2006]; Robinson-Smith T. M., et al., Cancer Res. 67(12):5708-16, 2007; Gazzaniga S., et al., J Invest Dermatol., 127(8):2031-41, 2007).

However, liposomal formulations have been found to cause hypersensitivity reactions in many patients, causing symptoms such as dyspnea, tachypnea, tachcardia, hypotension, hypertension, chest pain, back pain and other signs of cardiopulmonary distress (Chanan-Khan et al., Ann Oncol. 14:1430-7 [2003]; Cesaro et al., Support Care Cancer, 7: 284-6 [1999]; Weiss et al., J. Clin Oncol., 8: 1263-8 [1990]). Such reactions can be life threatening and frequently necessitate cessation of treatment with liposomal formulations or the use of suboptimal dosing regimens.

Accordingly, there is a need in the art for therapeutic compositions against tumors and/or tumor metastases, including compositions that can offer safe and effective alternatives to the use of liposomal formulations.

### BRIEF SUMMARY

Methods are provided herein for treating or preventing the growth, invasion and/or metastasis of a tumor by administering a composition comprising a bisphosphonate and a pharmaceutically acceptable carrier to a subject who has a tumor or who is at risk for developing a tumor, wherein the pharmaceutically acceptable carrier comprises non-liposomal particles.

In some aspects, the non-liposomal particles are suitable for uptake by cancer-associated macrophages. The non-liposomal particles can be spheroid particles in some aspects, or non-spheroid particles in other aspects. In various aspects, the non-liposomal particles have a mean diameter between about 10 nm and about 10,000 nm, or between about 20 nm and about 1000 nm, or between about 50 nm and about 500 nm. In some aspects, at least 90% of the non-liposomal particles have a diameter between about 20 nm and about 1000 nm.

In some aspects, the bisphosphonate is released as a free compound from the composition upon uptake of the non-liposomal particles by a cancer-associated macrophage. In further aspects, the bisphosphonate is non-covalently associated with the non-liposomal particles.

In some aspects, the compositions are administered intravenously. In other aspects, the compositions are administered directly to a tumor.

In some aspects, the compositions are administered in an amount effective to reduce the number of cancer-associated macrophages in the subject. In further aspects, the compositions are administered in an amount effective to reduce the number of cancer-associated macrophage progenitor cells in the subject.

In some aspects, the compositions have a lower affinity for bone than the free bisphosphonate compound. In further aspects, the non-liposomal particles comprising the compositions do not bear a tumor targeting ligand.

### BRIEF DESCRIPTION OF THE DRAWINGS

FIG. 1 is a graph of the particle size distribution of clodronate (5.6 mg/mL): hydroxyapatite (2% wt/wt) particles. Size distribution was determined by laser light scattering using a Malvern Zetasizer, with a measured zeta potential of −34.6 at pH 7.17.

FIG. 2 is a graph showing the effect of clodronate (5.6 mg/mL): hydroxyapatite (2% wt/wt) particles on the tumor volume growth of 4t1-luc cancer cells in a syngeneic mouse model of breast cancer. Clodronate (5.6 mg/mL): hydroxyapatite (2% wt/wt) particles were administered chronically; six administrations, twice a week starting on day 1 after tumor challenge. Tumor volume was determined from caliper measurements. Error bars are the standard error of the mean. Clodronate (5.6 mg/mL): hydroxyapatite (2% wt/wt) particles inhibited the growth of 4 μl tumors relative to PBS control, with a significant difference (P<0.05) at days 27 and 34.

FIG. 3 is a graph showing the effect of clodronate (5.6 mg/mL) on the tumor volume growth of 4t1-luc cancer cells in a syngeneic mouse model of breast cancer. Clodronate was administered chronically; six administrations, twice a week starting on day 1 after tumor challenge. Tumor volume was determined from caliper measurements, and error bars are the standard error of the mean. Clodronate (5.6 mg/mL) in PBS did not inhibit the growth of 4 μl tumors relative to PBS control.

FIG. 4 is a graph of the particle size distribution of pamidronate (2.5 mg/mL): hydroxyapatite (2% wt/wt) particles. Size distribution was determined by laser light scattering using a Malvern Zetasizer, with a measured zeta potential of −4.45 mV at a pH 5.67.

FIG. 5 is a graph showing the effect of pamidronate (2.5 mg/mL): hydroxyapatite (2% wt/wt) particles on the viability of RAW 264.7 cells. Pamidronate-HAP particles significantly decreased the viability of RAW 264.7 cells relative to equal concentrations of pamidronate in solution.

FIG. 6 is a graph showing the effect of pamidronate (2.5 mg/mL): hydroxyapatite (2% wt/wt) particles on the tumor volume growth of 4t1-luc cancer cells in a syngeneic mouse model of breast cancer. Pamidronate (2.5 mg/mL)-hydroxyapatite(2%) particles were administered chronically; six administrations, twice a week starting on day 1 after tumor challenge. Tumor volume was determined from caliper measurements. Error bars are the standard error of the mean. Pamidronate (2.5 mg/mL)-hydroxyapatite(2%) particles inhibited the growth of 4 μl tumors relative to PBS control, with a significant difference (P<0.05) at day 27.

FIG. 7 is a graph of the particle size distribution of alendronate (5 mg/mL): hydroxyapatite(2%) particles. Size distribution was determined by laser light scattering using a Malvern Zetasizer.

FIG. 8 is a graph of the particle size distribution of alendronate: calcium carbonate particles. Size distribution was determined by laser light scattering using a Malvern Zetasizer, with a measured zeta potential of +22.1 mV at a pH 8.10.

FIG. 9 is a graph showing the effect of alendronate: calcium carbonate particles on the viability of RAW 264.7 cells. Alendronate: calcium carbonate particles significantly decreased the viability of RAW 264.7 cells relative to equal concentrations of alendronate in solution.

### DETAILED DESCRIPTION

Descriptions of the invention are presented herein for purposes of describing various aspects, and are not intended to be exhaustive or limiting, as the scope of the invention will be limited only by the appended claims. Persons skilled in the relevant art can appreciate that many modifications and variations are possible in light of the aspect teachings.

Unless defined otherwise, all technical and scientific terms used herein have the meaning commonly understood by one of ordinary skill in the art to which this invention belongs. While exemplary methods and materials are described herein, it is understood that methods and materials similar or equivalent to those described can be used. All publications mentioned herein are incorporated by reference to disclose and describe the methods and/or materials in connection with which they are cited.

In various aspects, methods are provided for treating and/or preventing the growth of tumors and/or tumor metastases by administering a composition comprising a bisphosphonate and a particulate carrier to a patient that has or is at risk of developing tumors and/or tumor metastases. As described herein, it has been discovered that administering bisphosphonates in association with a non-liposomal particulate carrier can effectively deplete cancer associated myeloid derived cells, resulting in a reduction of cancerous growth. Also provided herein are compositions useful in depleting phagocytic cells that promote the growth, spreading, malignancy and/or metastasis of cancerous cells. Administering the compositions to an animal, preferably a human, in an effective amount depletes, inactivates and/or inhibits cancer associated myeloid derived cells.

Bisphosphonates administered according to the instant methods are formulated so that they enter cancer associated myeloid derived cells. For example, the bisphosphonates may be embedded, covalently linked, or adsorbed to the surface of a non-liposomal particle, preferably of a specific size, size range, or size distribution that allows the particles to enter cells primarily via phagocytosis. When so formulated, the bisphosphonate specifically targets and is efficiently engulfed by cancer associated myeloid derived cells. While cancer associated myeloid derived cells are characterized by a capacity to phagocytose particles, it is understood that other cell processes could be employed by targeted macrophages to take up particles, such as but not limited to, endocytosis, receptor-mediated endocytosis, and cell fusion.

Without being limited by a particular theory, it is believed that methods and compositions provided herein can exert an antitumor effect by depleting and/or inactivate cancer-promoting cells of myeloid origin which provide support for cancerous cells to proliferate locally and/or regionally, and/or to metastasize. In particular, particles taken up by cancer associated myeloid derived cells release the bisphosphonate, which advantageously inactivates or destroys the cell and/or modulates one or more cancer-promoting activities, such as the secretion of growth stimulating factors needed for angiogenesis and/or immune suppressing cytokines, thus suppressing the tumor stromal support needed for cancerous cells to proliferate. The proliferation of cancerous cells is thereby decreased and the ability of external or internal agents to cause cancerous cell destruction is enhanced, resulting in decreased proliferation and/or spreading or malignant or metastasized cancerous cells.

It must be noted that, as used in the specification, the singular forms “a,” “an” and “the” include plural referents unless the context clearly dictates otherwise.

The term “subject” is understood to include any animal, including but not limited to a human or a veterinary subject, such as a primate, a dog, a cat, a horse, a cow, and the like

The term “cancer associated myeloid derived cell” refers to cells typically of myeloid origin that have the capability of phagocytosis and which influence, directly and/or indirectly, the growth, proliferation, spread, and/or metastasis of cancerous cells. Such cells include, but are not limited to, tumor associated macrophages (e.g., macrophages that reside in the tumor stroma and produce signaling molecules that enhance the growth of cancerous cells and/or enable the spreading and/or metastases of tumor cells), as well as fibroblasts, neutrophils, resident macrophages, and dendritic cells of myeloid and non-myeloid origins. In various aspects, compositions provided herein are capable of targeting and depleting cancer associated myeloid derived cells residing within and/or surrounding a tumor, as well as cancer associated myeloid derived cells external to the tumor. For example, in some aspects, cancer associated myeloid derived cells targeted by methods provided herein include, e.g., Kupfer cells, myeloid derived spleen cells and/or circulating monocytes which indirectly influence tumor growth, proliferation, spread, and/or metastasis by, e.g., migrating to and entering the tumor, where they are converted to tumor associated macrophages. In further aspects, cancer associated myeloid derived cells targeted by the methods provided herien include resident macrophages and/or other phagocytic cells within non-cancerous tissues (e.g., tissues prone to metastatic cancer), which cells are capable of promoting the growth, proliferation, spread, and/or further metastasis of metastatic tumor cells that migrate to the tissue. Advantageously, administering a particulate bisphosphonate composition according to a method provided herein results in a sustained depletion of multiple forms and/or sources of cancer associated myeloid derived cells, leading to enhanced efficacy, fewer side effects, lower effective dosages, less frequent dosing, and/or other therapeutic benefits relative to other methods and compositions.

While the ability to phagocytose particulate matter is a defining characteristic of cancer associated myeloid derived cells, it is also acknowledged that targeted cells may use multiple processes to take up compositions provided herein, including but not limited to endocytosis, receptor mediated endocytosis, and cell fusion. Cancer associated myeloid derived cells can influence the growth, proliferation, spread, and/or metastases of cancerous cells via various mechanisms, including but not limited to, inhibiting secretion of growth promoting cytokines (e.g., those that induce angiogenesis and/or suppress the activity of cytotoxic T-cells and/or NK cells), secretion of chemokines, secretion of pro-angiogenic factors, secretion of cellular matrix degrading molecules, and/or suppression of cytotoxic immune responses against cancerous cells. Cancer associated myeloid derived cells can be of various phenotypes, including but not limited to, macrophages, dendritic cells, monocytes, and the like. Cancer associated myeloid derived cells may be variously referred to in the current literature by terms including but not limited to “tumor associated macrophages,” “tumor associated dendritic cells,” “dendritic cells,” “myeloid derived suppressor cells,” and “M2 macrophages.”

In some aspects, cancer associated myeloid derived cells include monocyte precursor cells that are produced in the bloodstream and extravasate into surrounding tissues, including malignant tumors, where they differentiate into macrophages and perform the immune, secretory, phagocytic and other functions of macrophages.

The term “depleting” as used herein with respect to cancer associated myeloid derived cells, means a relative reduction in the number and/or activity of cancer associated myeloid derived cells. For example, in some aspects, cancer associated myeloid derived cells are depleted upon administration of a particulate bisphosphonate composition relative to an earlier point in time control, such as, the untreated tumor.

Any bisphosphonate compound can be used in the particle compositions described herein. In some aspects, the bisphosphonate is a compound according to Formula I:

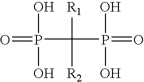

wherein,

R₁ is H, OH or a halogen atom; and

R₂ is halogen; linear or branched C₁-C₁₀ alkyl or C₂-C₁₀ alkenyl, each optionally substituted by heteroaryl, heterocyclyl, C₁-C₁₀ alkylamino, or C₃-C₈ cycloalkylamino, where the amino may be a primary, secondary or tertiary —NHY, where Y is hydrogen, C₃-C₈ cycloalkyl, aryl or heteroaryl; or

R₂ is -SZ where Z is chloro substituted phenyl or pyridinyl.

In some aspects, R₁ is preferably H or OH.

In further aspects, R₂ is preferably C₁-C₁₀ alkylamino, or C₃-C₈ cycloalkylamino, where the amino may be a primary, secondary or tertiary —NHY, where Y is hydrogen, C₃-C₈ cycloalkyl, aryl or heteroaryl.

In some aspects, R₁ is OH, and R₂ is CH₃ (etidronate).

In some aspects, R₁ is OH, and R₂ is CH₂CH₂NH₂ (pamidronate).

In some aspects, R₁ is OH, and R₂ is CH₂CH₂N(CH₃)₂ (dimethyl pamidronate).

In some aspects, R₁ is OH, and R₂ is CH₂CH₂CH₂NH₂ (alendronate).

In some aspects, R₁ is OH, and R₂ is CH₂CH₂N(CH₃)CH₂CH₂CH₂CH₂CH₃ (ibandronate).

In some aspects, R₁ is OH, and R₂ is CH₂-3-pyridine (risedronate).

In some aspects, R₁ is OH, and R₂ is CH₂-(1H-imidazole-1-yl) (zoledronate).

In some aspects, R₁ is H, and R₂ is CH₂—S-phenyl-Cl (tiludronate).

In some aspects, R₁ and R₂ are Cl (clodronate).

In some aspects, the bisphosphonate is not clodronate.

Bisphosphonates can be classified as simple bisphosphonates or as amino-bisphosphonates. The simple bisphosphonates are metabolized to non-hydrolysable analogs of adenosine triphosphate and diadenosine tetraphosphate within cells (Rogers M J et al., Biochem. Biophys. Res. Comm. 189:414-423 [1992]; Frith J C et al., J. Bone Min. Res. 12:1358-1367 [1997]), whereas the amino-bisphosphonate inhibit farnesyl diphosphate synthase, the major enzyme of the mevalonate pathway (Rogers M J, Calc. Tiss. Int. 75(6):451-461 [2004]).

In some aspects, the bisphosphonate is selected from the group consisting of: clodronate, alendronate, etidronate, tiludronate, pamidronate, ibandronate, neridronate, zoledronate, minodronate, and risedronate.

In some aspects, the bisphosphonate is an amino-bisphosphonate selected from the group consisting of: tiludronate, alendronate, pamidronate, ibandronate, neridronate, risedronate, zoledronate, and derivatives thereof.

In some aspects, the bisphosphonate is a simple bisphosphonate selected from the group consisting of: clodronate, etidronate, and derivatives thereof. In some aspects, the simple bisphosphonate is preferably etidronate or a derivative thereof.

Examples of additional bisphosphonates that can be used in methods and compositions provided herein include, but are not limited to, 3-(N,N-dimethylamino)-1-hydroxypropane-1,1-diphosphonic acid (dimethyl-APD); 6-amino-1-hydroxyhexane-1,1-di-phosphonic acid (amino-hexyl-BP); 3-(N-methyl-N-pentylamino)-1-hydroxy-propane-1,1-diphosphonic acid (methyl-pentyl-APD); 3-[N-(2-phenylthioethyl)-N-methylamino]-1-hydroxy-ypropane-1,1-bishosphonic acid; 1-hydroxy-3-(pyrrolidin-1-yl)propane-1,1-bisphosphonic acid; 1-(N-phenylaminothiocarbonyl)methane-1,1-diphosphonic acid (FR 78844 (Fujisawa)); 5-benzoyl-3,4-dihydro-2H-pyrazole-3,3-diphosphonic acid tetraethyl ester (U81581 (Upjohn)); 1-hydroxy-2-(imidazo[1,2-a]pyridin-3-yl)ethane-1,1-diphosphonic acid (YM 529); 2-(2-aminopyrimidinio) ethylidene-1,1-bisphosphonic acid betaine (ISA-13-1).

Bisphosphonates and other active compounds described herein include any pharmaceutically acceptable salts, derivatives, analogs, prodrugs, and metabolites of the compound, and in the case of compounds containing a chiral center, all possible stereoisomers of the compound. Compositions described herein can comprise a racemic mixture of two enantiomers or an individual enantiomer substantially free of the other enantiomer. If the named compound comprises more than one chiral center, compositions described herein may also include mixtures of varying proportions of the diastereomers, as well as one or more diastereomers substantially free of one or more of the other diastereomers. By “substantially free” it is meant that the composition comprises less than 25%, 15%, 10%, 8%, 5%, 3%, or less than 1% of the minor enantiomer or diastereomer(s). Methods for synthesizing and isolating stereoisomers are known in the art.

In various aspects, bisphosphonates administered according to methods provided herein are formulated with a pharmaceutically acceptable carrier. The pharmaceutically acceptable carrier can comprise any substance or vehicle suitable for delivering a bisphosphonate to a therapeutic target when the composition is administered according to a method provided herein. In some preferred aspects, the carrier is suitable for delivering an associated bisphosphonate into contact with cancer associated myeloid derived cells. In further aspects, the carrier is suitable for being phagocytosed by cancer associated myeloid derived cells, thus introducing the associated bisphosphonate to the intracellular space of the cancer associated myeloid derived cells.

In some preferred aspects, the carrier is a non-liposomal particle. The bisphosphonates and/or additional active agents of the compositions provided herein can be associated with the non-liposomal particles by any means. For example, the bisphosphonate can be encapsulated, entrapped, embedded, adsorbed within the particle, dispersed in a particle matrix, adsorbed or linked on the particle surface, covalently linked to a particle matrix, or a combination thereof, and can be dispersed uniformly or non-uniformly on the surface and/or within the particles.

In some aspects, the particles comprise a particulate matrix capable of being formed into a specific dimension. In some aspects, the particles comprise one or more shapes or geometries that facilitate selective phagocytosis by cancer associated myeloid derived cells. The particle matrix can include, but is not limited to, inorganic materials, polymers, polypeptides, proteins, lipids, and surfactants, and can be formed into nanospheres, nanoparticles, microcapsules, nanocapsules, microspheres, microparticles, colloids, aggregates, flocculates, insoluble salts, emulsions and insoluble complexes (e.g. M. Donbrow in: Microencapsulation and Nanoparticles in Medicine and Pharmacy, CRC Press, Boca Raton, Fla. 347, 1991).

The particle matrix can comprises polymeric and/or non-polymeric materials, and is preferably biocompatible and/or biodegradable. In some preferred aspects, the particulate matrix is comprised of a biodegradable polymer, such as poly(lacto-co-glycolide) (PLG), poly(lactide), poly(caprolactone), poly(hydroxybutyrate), poly(beta-amino) esters and/or copolymers thereof. Alternatively, the particles can comprise other materials, including but not limited to, poly(dienes) such as poly(butadiene) and the like; poly(alkenes) such as polyethylene, polypropylene and the like; poly(acrylics) such as poly(acrylic acid) and the like; poly(methacrylics) such as poly(methyl methacrylate), poly(hydroxyethyl methacrylate), and the like; poly(vinyl ethers); poly(vinyl alcohols); poly(vinyl ketones); poly(vinyl halides) such as poly(vinyl chloride) and the like; poly(vinyl nitriles), poly(vinyl esters) such as poly(vinyl acetate) and the like; poly(vinyl pyridines) such as poly(2-vinyl pyridine), poly(5-methyl-2-vinyl pyridine) and the like; poly(styrenes); poly(carbonates); poly(esters); poly(orthoesters); poly(esteramides); poly(anhydrides); poly(urethanes); poly(amides); cellulose ethers such as methyl cellulose, hydroxyethyl cellulose, hydroxypropyl methyl cellulose and the like; cellulose esters such as cellulose acetate, cellulose acetate phthalate, cellulose acetate butyrate, and the like; poly(saccharides), protein, polypeptides, gelatin, starch, gums, resins and the like. These materials may be used alone, as physical mixtures (blends), or as copolymers.

In some aspects, the particle matrix comprises a water-insoluble inorganic material, such as hydroxyapatite, calcium phosphate, calcium carbonate, calcium oxide, or the like, optionally containing one or more additional inorganic elements such as magnesium, beryllium, barium, copper, gallium, iron, gadolinium, silicon, zinc, nickel, cobalt or other cationic ion either used in combination with calcium or as substitutes for calcium within the particle.

In further aspects, the particulate matrix is comprised of lipids both in the fluid or solid phase and are constituted of mono-, di- and triglycerides of short, medium and long chain fatty acids; hydrogenated vegetable oils; fatty acids and their esters; fatty alcohols and their esters and ethers; natural or synthetic waxes; wax alcohols and their esters; sterols; hard paraffins; or mixtures of the above-mentioned lipids with the resulting particulate either an emulsion or a solid lipid particle.

In some aspects, the non-liposomal particles are comprised of an inorganic solid, such as gold, silica, or the like, and the bisphosphonate is adsorbed on the surface of the particle.

Upon being administered to a cell or tissue, compositions provided herein preferably maintain bisphosphonates in stable association with the non-liposomal particle carrier in vivo until the composition contacts and is phagocytosed by a cancer associated myeloid derived cell. In some aspects, non-liposomal particles used herein selectively release bisphosphonates within a cancer associated myeloid derived cell upon being phagocytosed. For example, in some aspects, a non-liposomal particle has greater affinity for an associated bisphosphonate within the systemic circulation and/or within other in vivo environments than within cancer associated myeloid derived cells targeted for treatment, so that the bisphosphonate is selectively released within the cancer associated myeloid derived cells. In further aspects, a bisphosphonate may remain associated with the non-liposomal particle upon being phagocytosed by a cancer associated myeloid derived cell, for example where the bisphosphonate can exert a therapeutic effect against the cancer associated myeloid derived cell while associated with the non-liposomal particle.

In some preferred aspects, the non-liposomal particle carriers provided herein have one or more properties that allow the carriers, as well as bisphosphonates and other active agents associated with the carriers, to be efficiently phagocytosed by cancer associated myeloid derived cells. For example, in some aspects, non-liposomal particles used herein have a size, shape, solubility, and/or charge that allows bisphosphonate compositions to be readily phagocytosed by cancer associated myeloid derived cells. In various embodiments, compositions provided herein comprise particles having a diameter or width between about 10 nm and about 10 μm, or preferably between about 20 nm and 1 μm, or more preferably between about 50 nm and about 500 nm. In further aspects, compositions provided herein comprise particles having a size distribution that allows a significant portion of a population of such particles administered to a patient to be phagocytosed by cancer associated myeloid derived cells. For example, in some aspects, compositions provided herein comprise non-liposomal particles, at least 90% of which have a diameter between about 10 nm and about 10000 nm, or preferably between about 20 nm and about 1000 nm, or more preferably between about 50 nm and about 500 nm. The sizes of particles comprising the compositions provided herein can be determined using any method known in the art, such as laser light scattering.

In further aspects, non-liposomal particle carriers used in methods provided herein have one or more properties that allow the carriers, as well as bisphosphonates and other active agents associated with the carriers, to be selectively phagocytosed by cancer associated myeloid derived cells. For example, macrophages, in contrast to many other cell types, have the ability to phagocytose particles up to about 20 μm in diameter (Cannon G J et al., J. Cell Sci. 101:907-913 [1992]), while also preferentially taking up particles having an average size as small as 40 nm (Ong T H et al., J. Nanopart. Res. 10(1):141-150 [2008]). Accordingly, in some aspects, compositions provided herein comprise non-liposomal particles having a size and/or size distribution that allows the particles to be selectively phagocytosed by cancer associated myeloid derived cells. For example, in some aspects, compositions provided herein have a diameter or width between about 70 nm and about 300 nm, or more preferably between about 100 nm and 180 nm. Advantageously, selectively phagocytosis of compositions provided herein allows bisphosphonates to be delivered primarily to the interior of cancer associated myeloid derived cells, minimizing cytotoxicity to non-phagocytic cells. In some aspects, administering a bisphosphonate particle composition provided herein exposes cancer associated myeloid derived cells to a therapeutic level of the bisphosphonate, while exposing other non-targeted cells, tissues, and/or organs to sub-therapeutic levels of the bisphosphonate.

In further aspects, compositions provided herein comprise non-liposomal particles having a multi-modal size distribution that allows the particles to be readily phagocytosed by two or more types of cancer associated myeloid derived cells. For example, compositions provided herein may comprise particles having a bimodal size distribution, where particles of a first size range are more efficiently phagocytosed by a first population of cancer associated myeloid derived cells and particles of a second size range are more efficiently phagocytosed by a second population of cancer associated myeloid derived cells. In some aspects, the first and second populations of cancer associated myeloid derived cells include circulating monocytes and tumor-associated macrophages.

In some aspects, two or more types of cancer associated myeloid derived cells can be targeted by formulating a bisphosphonate with two or more particle types having different characteristics, such as size and/or size distribution.

In further aspects, the bisphosphonate active agent itself is formulated in a particulate form having one or more properties suitable for efficient phagocytosis by cancer associated myeloid derived cells. For example, in some aspects, a the bisphosphonate is combined with a flocculating agent, such as Cetyl trimethylammonium bromide (a.k.a. hexadecyl trimethyl ammonium bromide, and other alkyltrimethylammonium salts), cetylpyridinium chloride, polyethoxylated tallow amine, benzalkonium chloride, benzethonium chloride and the like, which complexes with the bisphosphonate to form flocculant bisphosphonate particulates of a desired size.

Non-liposomal particles described herein can include suspending agents, stabilizers, and/or other pharmaceutically acceptable excipients. Suitable excipients include any excipients or formularies useful for in vivo delivery, including, e.g., water, phosphate buffered saline, Ringer's solution, dextrose solution, serum-containing solutions, Hank's solution, other aqueous physiologically balanced solutions, oils, esters and glycols. Aqueous carriers can contain suitable auxiliary substances required to approximate the physiological conditions of the recipient, for example, by enhancing chemical stability and isotonicity. Alternatively, or in addition, aqueous carriers can contain cryoprotective agents that can preserve the integrity of the particles upon reconstitution of a frozen and/or lyophilized composition of bisphosphonates particles.

In further aspects, the surface of non-liposomal particles is coated or embedded with surface agents capable of enhancing the phagocytosis by cancer associated myeloid derived cells. For example, in some aspects, non-liposomal particles are coated with surface agents that confer a net cationic, anionic, zwiterionic surface charge, and/or agents that bind to receptors on the targeted phagocyte. In some preferred aspects, the surface agent is mannan or mannose that binds to the mannose receptor preferentially expressed by M2 phenotype macrophages. In further aspects, the surface agents preferentially bind to Scavenger Receptor A, Scavenger Receptor B, CD163, CD14, CD23, or a combination thereof.

Compositions provided herein can be formulated using any of the conventional techniques known in the art (see, for example, Remington's Pharmaceutical Sciences, Chapter 43, 14th Ed., Mack Publishing Co, Easton, Pa. 18042 USA). The formulations may be prepared in forms suitable for injection, infusion, instillation or implantation in the body, such as, for example, a suspension of particles. For example, compositions provided herein may be formulated with appropriate pharmaceutical additives for parenteral dosage forms. The preferred administration form in each case depends on the desired delivery mode, which is usually that which is the most physiologically compatible with the patient's condition, the extent of cancer cell proliferation and migration as well as other possible therapeutic treatments, anti-neoplastic agents or immunotherapeutic agents, used to reduce the cancerous cell burden within that individual.

In various aspects, methods are provided herein for treating and/or preventing tumor growth, regionally spreading cancer, and/or tumor metastases by administering to an animal, preferably a human, an effective amount of a formulation comprising a bisphosphonate formulated with a non-liposomal particle. In some preferred aspects, the particles have one or more properties that facilitates phagocytosis by cancer associated myeloid derived cells, such as but not limited to, particle size, which allows the formulation to be taken-up by the phagocytic cells causing inhibition and/or destruction of the phagocytic cells.

The term “effective amount” denotes an amount of a formulation provided herein which is effective in achieving a desired therapeutic result, such as an adjuvant treatment of cancer or one or more physiological effects associated with the treatment of cancer. In various aspects, compositions provided herein are administered in an amount effective to i) inhibit and/or decrease the phagocytic activity of cancer associated myeloid derived cells, ii) inhibit and/or decrease the secretion of factors by cancer associated myeloid derived cells that promote angiogenesis and/or tumor cell proliferation, migration, and/or metastasis; and/or iii) eliminate and/or decrease the number of macrophages/monocytes in circulation. For example, without being bound by any particular theory, it is believed that bisphosphonates can, inter alia, reduce the ability of cancer associated myeloid derived cells to produce and/or shed chemoattractants, chemokines and angiogenesis promoting factors. In particular, delivery of the bisphosphonates into the interior of the cell induces the phagocytic cell to undergo apoptosis and cell death.

Phagocytic activity can be assayed by the level of cell activation in response to an activating stimulus. For example, macrophage/monocyte activation can be assayed by quantifying the levels of chemotactic factors, such as macrophage chemoattractant protein-1 (MCP-1) and macrophage inflammatory protein-1 alpha (MIP-1 alpha). The levels of various factors produced by macrophages, such as interleukin 1 beta (IL-1β), tissue necrosis factor alpha (TNF-α), histamine, tryptase, PAF, and eicosanoids such as TXA₂, TXB₂, LTB₂, LTB₄, LTC₄, LTD₄, LTE₄, PGD₂ and TXD₄, can be assayed by any suitable method known in the art, including but not limited to, ELISA, immunoprecipitation, and quantitative western blot. The number of cancer associated myeloid derived cells can be assayed by measuring cell proliferation, e.g., by measuring 3H-thymidine incorporation, by direct cell count, by detecting changes in transcriptional activity of known genes, such as proto-oncogenes (e.g., fos, myc) or cell cycle markers, or by trypan blue staining. Any suitable method known in the art can be used to assay for levels of mRNA transcripts (e.g., by northern blots, RT-PCR, Q-PCR, etc.) or protein levels (e.g., ELISA, western blots, etc.).

An effective amount will of course depend on a number of factors in any given case, including but not limited to, weight and gender of the treated individual, mode of administration (e.g., whether it is administered systemically or directly to the site), therapeutic regime (e.g. whether the formulation is administered once daily, several times a day, once every few days, or in a single dose), clinical indicators of cancer spread, and whether the cancer has spread regionally, spread to the lymph nodes or metastasized to other tissues. Skilled artisans, by routine experimentation, can readily determine an effective amount in each case.

In various aspects, methods provided herein ameliorate, alleviate, and/or eliminate a condition targeted for treatment (e.g., a cancer or other form of neoplastic cell growth), or one or more symptoms and/or indicators of a condition targeted for treatment. For example, methods provided herein may cause tumor regression, reduction in tumor weight and/or size, increased time to progression, enhanced duration of survival, enhanced progression free survival, enhanced overall response rate, enhanced duration of response, enhanced quality of life, and/or enhanced overall well being, as measured by objective and/or subjective criteria. In some aspects, compositions provided herein inhibit metastatic spread without detectable shrinkage of a primary tumor. In further aspects, compositions provided herein exert a tumoristatic effect without detectably reducing tumor size.

Methods and compositions provided herein are useful for treating a variety of tumors. For example, the origin of the tumor can be breast cancer, ovarian cancer, gynecological cancer, hepatobiliary cancer, colorectal cancer, prostate cancer, lung cancer, pancreatic cancer, kidney cancer, bladder cancer, melanoma, malignant lymphoma and central nervous system cancer, head and neck cancer, or a tumor metastasis originating from any of said tumors.

In some aspects, the tumor or metastasis is not a bone tumor or metastasis.

Methods and compositions provided herein are also useful for treating a variety of cancers. Examples of cancers that may benefit from the depletion of cancer associated myeloid derived cells include, but are not limited to, fibrosarcoma, myxosarcoma, liposarcoma, chondrosarcoma, osteogenic sarcoma, chodoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillay adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oliodendroglioma, meningioma, melanoma, neuroblastoma, retinoblastoma, leukemia, lymphoma, multiple myeloma, Waldenstrom's macroglobulinemia, and heavy chain disease.

In some aspects, methods provided herein for treating cancer by depleting cancer associated myeloid derived cells prior to the administration of an immune stimulating agent. To deplete cancer associated myeloid derived cells, the methods involve administering an effective amount of a formulation to an animal, preferably a human, comprising a bisphosphonate in a non-liposomal particulate formulation. The formulation specifically targets phagocytic cells by virtue of its properties, such as but not limited to, size, which allows the formulation to be taken-up primarily by phagocytosis. Once the formulation is taken-up by cancer associated myeloid derived cells, the bisphosphonate is released causing inhibition and/or destruction of the phagocytic cells. The immune stimulating agent can either activate selective parts of the immune system or be an external agent designed to complement the host immune system to facilitate cancerous cell removal. An immune-stimulating agent can include but is not limited to a vaccine to stimulate T-cell destruction of cancerous cells, a re-infusion of the patient's own tumor-activated immune cells, adoptive cell transfer therapy, a competent or inactivated virus, a competent or inactivated bacteria infusion, and/or one or more immune stimulating molecules.

In some aspects, particulate bisphosphonate compositions provided herein enable treatment of therapeutic targets that are inaccessible or poorly accessible to free bisphosphonates and/or known bisphosphonate formulations. For example, the pharmacokinetics of many bisphosphonates are characterized by low levels of intestinal absorption and highly selective localization within bone. Without being limited by a particular theory, it is believed that bisphosphonates can associate with non-liposomal particle carriers described herein in a manner that reduces the exposure of the bisphosphonates to potential binding sites, such as hydroxyapatite, and/or to degradative factors, such as enzymes, hydrolyzing conditions, and the like. Thus, in some aspects, administering a bisphosphonate composition provided herein exposes therapeutic targets (e.g., cancer associated myeloid derived cells) to significantly higher effective concentration of the bisphosphonate than administering a comparable amount of the bisphosphonate as a free compound and/or as a known formulation. Advantageously, the higher effective bisphosphonate concentrations of compositions provided herein results in enhanced efficacy, fewer side effects, lower effective dosages, less frequent dosing, and/or other therapeutic benefits.

Compositions provided herein may be administered by any route which effectively transports the non-liposomal particle formulation to the appropriate or desirable site of action. Preferred modes of administration include intravenous (IV), intra-arterial (IA), and/or intratumoral (IT). Other suitable modes of administration include intradermal (ID), subcutaneous (SC), oral, interaperitoneal (IP), intrathecal, transdermal, transmucosal, and inhalation or bronchial instillation. Compositions may be administered, e.g., by bolus injections or infusions, and either directly or after dilution. Additional routes of administration and/or combinations of the above routes of administration may also be used depending on the desired therapeutic outcome, the type of tumor to be treated, the patient, and other considerations known to skilled artisans.

The dosage of compositions described herein will depend on a variety of factors, such as the specific activity of the agent selected, the mode of administration, the form of the formulation, the size of the formulation, the use of surface agents that may possibly enhance phagocytosis, and other factors as known per se. The non-liposomal particles may be prepared by any of the methods known within the art. The non-liposomal particles may include a surface charge or a surface ligand to enhance attachment and promote phagocytosis. Suitable particles in accordance with the invention are preferably non-toxic degradable particles in which the diameter of the particles range between about 0.01 and 10 microns, a diameter suitable for preferential phagocytosis by tumor associated macrophages and other phagocytes that promote cancerous cell proliferation. However other size ranges suitable for phagocyte uptake may also be used.

In further aspects, pharmaceutical compositions are provided comprising a bisphosphonate and a pharmaceutically acceptable carrier, the carrier comprising a non-liposomal particle, and one or more optional stabilizers, diluents, or excipients. The compositions are useful for treating cancer by effecting the depletion of cancer associated myeloid derived cells. Pharmaceutically acceptable carriers are know in the art, and include, e.g., aqueous isotonic solutions for sterile injectable compositions, which can contain antioxidants, buffers, bacteriostats and solutes that render the formulation isotonic with the blood of the intended recipient, and aqueous and non-aqueous sterile suspensions, which can include suspending agents, solubilizers, thickening agents, stabilizers, preservatives, microspheres or other agents to aid in the distribution and/or delivery of the bisphosphonate particles to targeted sites and/or targeted cancer associated myeloid derived cells.

In some aspects, methods provided herein may optionally further comprise administering one or more additional active agents. Examples of useful additional agents include, but are not limited to, an anti-neoplastic agent, an additional tumor stromal targeted therapy, and an immune modulator.

Classes of anti-neoplastic agents useful in combination with bisphosphonates include, but are not limited to, chemotherapeutic agents, growth inhibitory agents, cytotoxic agents, radiotherapy agents, apoptotic agents, toxins, and other cancer-treating agents known in the art, as well as combinations thereof. Examples of useful anti-neoplastic agents include, but are not limited to, anti-tubulins (e.g., vinca alkaloids, such as vincristine, vinblastine, vinflunine, vindesine, vinorelbine; taxanes, such as paclitaxel, docetaxel; epothilones); topo I inhibitors (e.g., camptothecins, such as topotecan, irinotecan, acetylcamptothecin, scopolectin, and 9-aminocamptothecin); topo II inhibitors (e.g., doxorubicin, detorubicin, epirubicin, esorubicin, idarubicin daunorubicin, etoposide (VP-16), and bleomycin); DNA alkylating agents (e.g., nitrogen mustards, such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosoureas, such as carmustine, chlorozotocin, fotemustine, lomustine, nimustine, and ranimnustine; alkyl sulfonates, such as busulfan, improsulfan and piposulfan; and aziridines, such as benzodopa, carboquone, meturedopa, and uredopa); anti-metabolites (e.g., methotrexate, gemcitabine, tegafur, capecitabine, epothilones, and 5-fluorouracil (5-FU)); folic acid analogues; pyrimidine analogs; antibiotics; and platinum analogs, as well as combinations thereof. In some aspects, the anti-neoplastic agent is a known formulation comprising a combination of two or more agents, such as CHOP (cyclophosphamide, doxorubicin, vincristine, and prednisolone) or FOLFOX (oxaliplatin, 5-FU, and leucovovin).

Tumor stromal targeted agents preferentially target stromal components that enable tumor growth and can include, e.g., anti-angiogenesis agents, hormonal agents (e.g., human growth hormone, parathyroid hormone, thyroxine, insulin, relaxin, and glycoprotein hormones such as follicle stimulating hormone (FSH), thyroid stimulating hormone (TSH), and luteinizing hormone (LH)), cytokines (e.g., growth factors, interferons, and interleukins), VEGF antagonists, epidermal growth factor receptor (EGFR) antagonists (e.g., tyrosine kinase inhibitors), platet-derived growth factor (PDGF) antagonists, stem cell factor (SCF), HER1/EGFR inhibitors, COX-2 inhibitors, ErbB2/3/4 antagonists, and other cancer-treating agents known in the art, as well as combinations thereof.

Classes of immune modulators useful in combination with bisphosphonates in compositions provided herein include, but are not limited to, monoclonal antibodies (e.g., anti-HER2 antibodies, such as trastuzumab; anti-CD receptor antibodies, such as rituximab and ibritumomab tiuxetan (CD20), gemtuzamab ozogamicin (CD33), and alemtuzumab (CD52)); anti-epidermal growth factor receptor (EGFR) antibodies, such as cetuximab; anti-vascular epidermal growth factor receptor (VEGFR) antibodies, such as bevacizumab; anti-tumor necrosis factor-beta antibodies; anti-interleukin-2 and anti-IL-2 receptor antibodies; anti-LFA-1 antibodies, such as anti-CD11a and anti-CD 18 antibodies), anti-inflammatory agents, nonsteroidal antiinflammatory drugs (NSAIDs), toll-like receptor (TLR) agonists, complement inhibitors, notch binding proteins, immunostimulatory agents, and other cancer-treating agents known in the art, as well as combinations thereof.

In some aspects, a composition provided herein comprises a known particulate anticancer agent or formulation, such as ABRAXANE™ (albumin-engineered nanoparticle formulation of paclitaxel), as a pharmaceutically acceptable carrier, which is modified to incorporate one or more bisphosphonates.

In some aspects, an anti-neoplastic agent, an additional tumor stromal targeted therapy, or an immune modulator reduces and/or eliminates cancerous cells directly to supplement effects mediated through the depletion and/or modulation of cancer associated myeloid derived cells. In further aspects, an additional active agent modulates the activity of a bisphosphonate of a composition provided herein. In some aspects, administering a composition comprising a bisphosphonate and an additional active agent enhances one or more aspects of treating and/or preventing tumor growth, invasion and/or metastasis relative to compositions comprising the bisphosphonate and the additional active agent individually. For example, in various aspects, administering a bisphosphonate in combination with an additional active agent can result in enhanced efficacy, fewer side effects, lower effective dosages, less frequent dosing, and/or other therapeutic benefits. In some preferred aspects, a composition comprising a bisphosphonate and an additional active agent enhances one or more aspects of treating and/or preventing tumor growth, invasion and/or metastasis in a synergistic manner.

The one or more additional active agents can comprise part of the same particle formulation as the bisphosphonate or a different formulation, which can be a particle formulation or a non-particle formulation. In some aspects, the one or more additional active agents comprises a different bisphosphonate. In further aspects, a bisphosphonate particle composition is administered in combination with a different formulation of the same bisphosphonate, which can be a particulate formulation having different properties as the primary formulation or a non-particulate formulation.

The bisphosphonate and the one or more additional agents need not be administered at exactly the same time, but rather are administered in a sequence and within a time interval such that they can act together to provide an increased benefit than if they were administered otherwise. For example, each therapeutic agent may be administered at the same time or sequentially in any order at different points in time; however, if not administered at the same time, they should be administered sufficiently close in time so as to provide the desired therapeutic effect. Each therapeutic agent can be administered separately, in any appropriate form and by any suitable route which effectively transports the therapeutic agent to the appropriate or desirable site of action.

Having now generally described the invention, the same will be more readily understood through reference to the following examples which are provided by way of illustration, and are not intended to be limiting of the disclosed invention, unless specified.

### EXEMPLARY ASPECTS

### Example 1

### Clodronate-Hydroxyapatite Nanoparticles Inhibit 4 μL Breast Cancer Tumor Growth

This example illustrates that clodronate-hydroxyapatite nanoparticles inhibit the growth of 4 μl breast cancer tumors in a mouse model

Preparation of Clodronate-Hydroxyapatite Nanoparticles.

Clodronate-hydroxyapatite nanoparticles were prepared by combining commercially available clodronate with a nanosuspension of hydroxyapatite nanoparticles using a variation on that previously described (Ong H T et al., J. Nanopart. Res. 10: 141-150, 2008). Briefly, clodronate (473 mgs, Sigma-Aldrich) was added to a suspension of filtered 4% (wt/wt) hydroxyapatite nanoparticles (40 mL, Himed). The suspension was allowed to incubate overnight to allow the clodronate to bind to the hydroxyapatite. The suspension was diluted with a 2× phosphate buffered saline (Sigma-Aldrich) for a final suspension of clodronate (5.6 mg/mL), hydroxyapatite nanoparticles (2 wt/wt %) in phosphate buffered saline.

Characterization of Clodronate-Hydroxyapatite Nanoparticles.

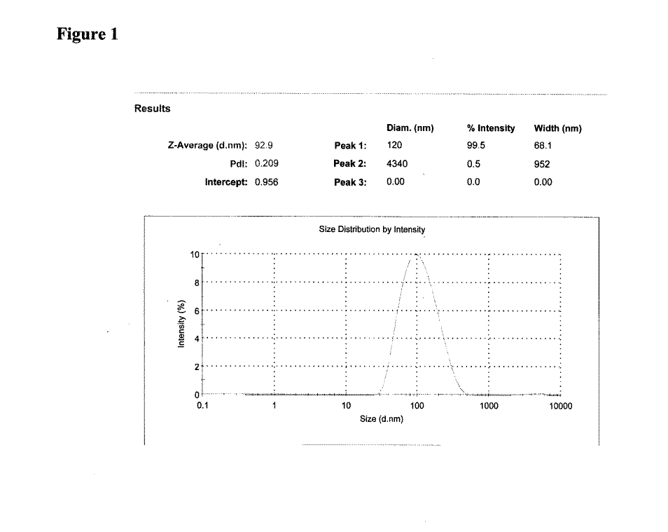

For the determination of particle size distribution, the clodronate-hydroxyapatite suspension was measured using a Malvern laser light scattering particle sizer. The results are depicted in FIG. 1.

Quantification of the amount of clodronate contained in the hydroxyapatite nanoparticles was determined by measuring the supernatant concentration of clodronate after incubation with the hydroxyapatite nanoparticles. Clodronate solution concentrations were determined using a mass spectrometer detector and a variation on the Fernandes et al. method (Fernandes C. et al., J. Chrom Sci 45:236-241, 2007). Measurement of the clodronate concentrations in the original solution and the supernatant from the particles demonstrated that 20% of the clodronate was associated with the HAP particles.

In Vitro Cell Cytotoxicity Assay of Clodronate-Hydroxyapatite Nanoparticles.

The effect of clodronate-hydroxyapatite nanoparticles on the cell viability of the cell line RAW 264.7 was determined using the MTT assay. In brief, cells were plated into 96-well plates at 5% confluence and incubated with formulation at full strength (10% in DMEM culture medium) for 5 days. Media was removed and replaced with fresh RPMI medium containing MTT. Half of the cells were lysed before MTT addition as a control for background absorbance. After 2 remaining unlysed cells were lysed and OD 560 was determined. Four replicates were run for each condition.

In Vivo Testing of Clodronate-Hydroxyapatite Nanoparticles in Breast Cancer Model.

In vivo testing was done with a syngeneic 4t1 model of breast cancer in immuno-competent Balb/c mice. This syngeneic in vivo model of cancer has the distinguishing characteristic of reliably modeling the processes of breast cancer tumor growth in an immuno-competent host. The effects of the clodronate-hydroxyapatite nanoparticle suspensions on the tumor growth were performed using methods previously described (Tao K et al., BMC Cancer 8:228, 2008). Briefly, primary tumors were established in mammary tissue of 6 week old Balb/c mice by injection of 106 4t1-12B cells subcutaneously under the nipple. The mice were kept in standard housing and with a normal diet. Each group of mice (6-10 per group, 20 g±10% body weight) were injected (100 microliters) 2 times per week through the tail vein for a period of 3 weeks, starting either on day one or day six after the tumor challenge. Clodronate solution was dissolved in PBS and given intravenously starting on day one after tumor challenge. Control mice were injected intravenously through the tail vein with PBS. Tumor growth was measured in a blinded fashion with a caliper each week and tumor volumes were calculated using the measured dimensions. Body weights were recorded weekly, and mice were sacrificed on day 34.

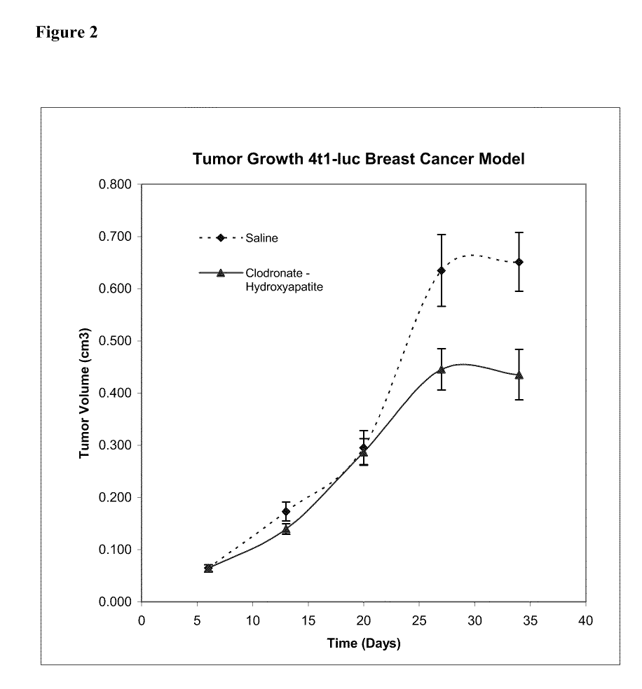

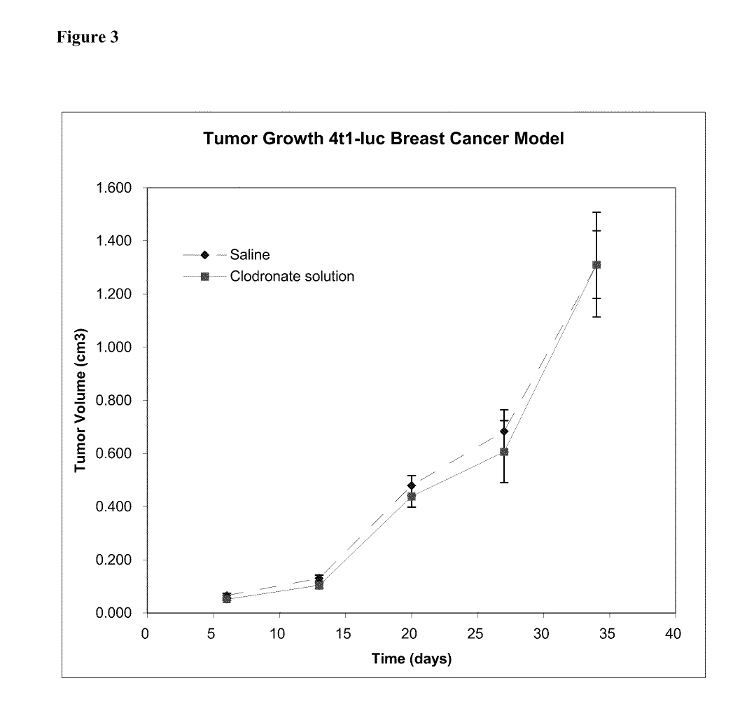

The results, depicted in FIG. 2 and FIG. 3, show that clodronate-hydroxyapatite significantly (P<0.05) inhibits growth of 4 μl tumors in vivo compared to PBS (FIG. 2) and that clodronate solution (5 mg/mL) dissolved in PBS is unable to inhibit growth of 4 μl tumors as effectively as clodronate-hydroxyapatite (FIG. 3).

### Example 2

### Pamidronate-Hydroxyapatite Nanoparticles Inhibit 4 μL Breast Cancer Tumor Growth

This example illustrates that pamidronate-hydroxyapatite nanoparticles inhibit the growth of 4 μl breast cancer tumors in a mouse model.

Preparation of Pamidronate-Hydroxyapatite Nanoparticles.

The preparation of the pamidronate-hydroxyapatite suspension was similar to the that used in Example 1 with the difference of 2.5 mg/mL pamidronate used in the suspension. Briefly, pamidronate (200 mg, Sigma-Aldrich) was added to 40 mL of hydroxyapatite nanoparticle suspension (4%, Himed). The suspension was allowed to incubate to allow time for the pamidronate to adsorb onto the hydroxyapatite. The pamidronate-hydroxyapatite suspension was diluted with 2×PBS to a final concentration of pamidronate (2.5 mg/mL)-hydroxyapatite(2%).

Characterization of Pamidronate-Hydroxyapatite Nanoparticles.

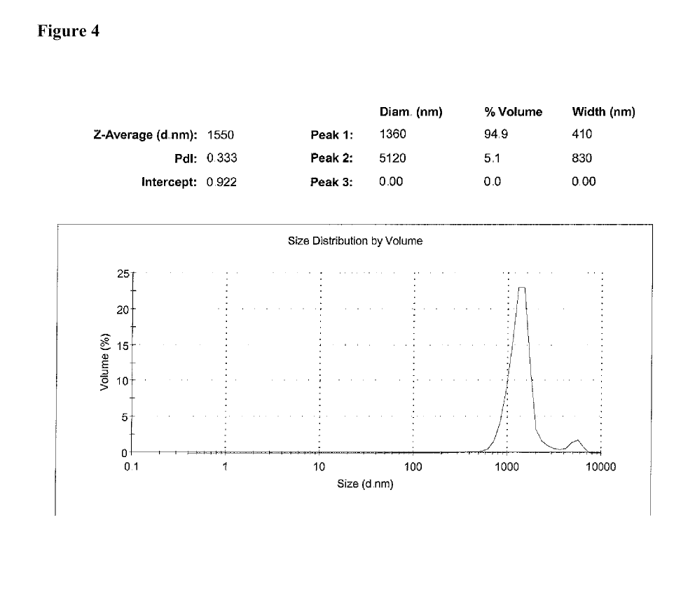

For the determination of particle size distribution and zeta potential, the pamidronate-hydroxyapatite suspension was measured using a Malvern laser light scatter particle sizer. The results are depicted in FIG. 4.

Quantification of the amount of pamidronate contained in the hydroxyapatite nanoparticles is determined by measuring the supernatant concentration of pamidronate after incubation with the hydroxyapatite nanoparticles. The assay for pamidronate is done using an indirect UV method (Fernandes C et al., J. Chrom. Sci. 45:236-241, 2007).

In Vitro Cell Cytotoxicity Assay of Pamidronate-Hydroxyapatite Nanoparticles.

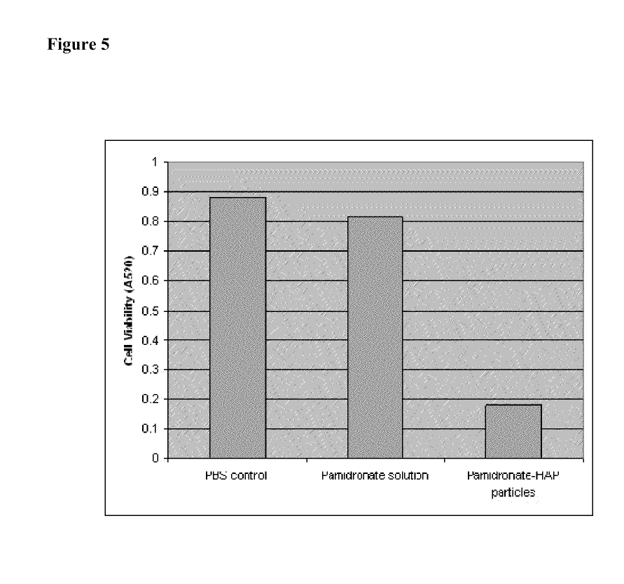

The effect of pamidronate-hydroxyapatite nanoparticles on the cell viability of the RAW 264.7 cell line was determined using the MTT assay. In brief, cells were plated into 96-well plates at 5% confluence and incubated with formulation at full strength (10% in DMEM culture medium) for 4 days. Media were removed and replaced with fresh RPMI medium containing MTT. Half of the cells were lysed before MTT addition as a control for background absorbance. After 2 remaining unlysed cells were lysed and OB 560 determined. Five replicates were run for each condition. Results are depicted in FIG. 5. Pamidronate-HAP particles significantly decreased the viability of RAW 264.7 cells relative to equal concentrations of pamidronate in solution.

In Vivo Testing of Pamidronate-Hydroxyapatite Nanoparticles in Breast Cancer Model.

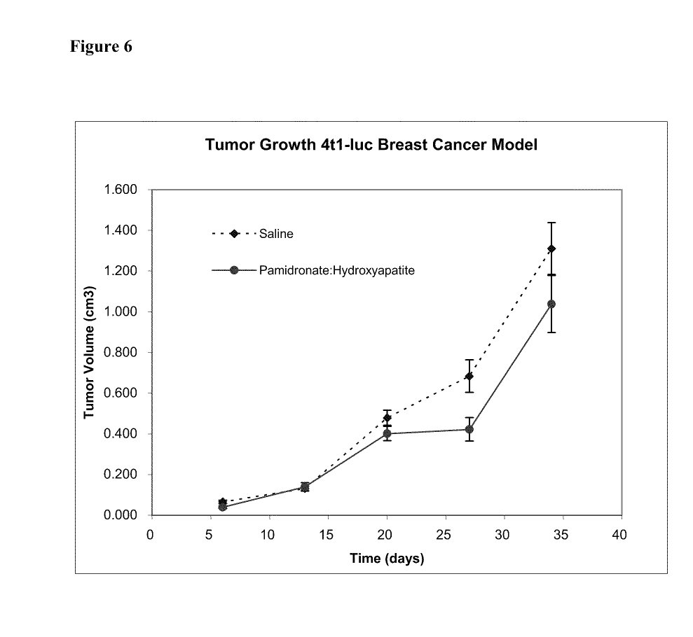

In vivo testing of pamidronate-hydroxyapatite nanoparticles was carried out essentially as described in Example 1. As depicted in FIG. 6, the treatment of 4 μl bearing Balb/c mice resulted in a significant reduction of tumor growth when compared to PBS controls as noted on day 27 of the experiment.

### Example 3

### Alendronate-Hydroxyapatite Nanoparticles

This example illustrates the preparation of an alendronate-hydroxyapatite nanoparticle suspension.

The preparation of the alendronate-hydroxyapatite suspension was similar to that used in Examples 2 and 3. Briefly, alendronate (100 mg, Sigma) was mixed with 10 mL of hydroxyapatite nanoparticle suspension (4%, Himed). The alendronate was allowed to incubate at room temperature for nine days with periodic mixing.

The percent alendronate bound to the hydroxyapatite nanoparticles was determined using a difference between the solution concentrations of alendronate initially and after the alendronate-hydroxyapatite suspensions were allowed to incubate. The concentrations were determined spectrophotometrically using a ninhydrin assay.

The percent of alendronate bound to the hydroxyapatite nanoparticles was determined to be 89% of the total alendronate yielding a mass ratio of alendronate to hydroxyapatite of 22%.

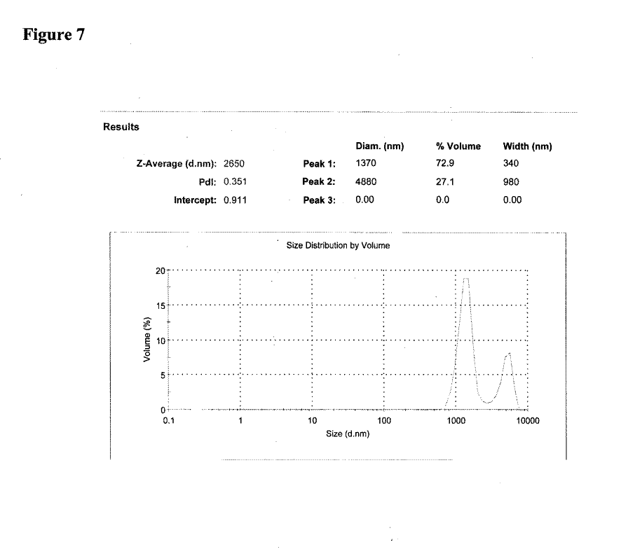

The particle size distribution of the alendronate-hydroxyapatite nanoparticles diluted in PBS is shown in FIG. 7.

### Example 4

### Alendronate-Calcium Carbonate Nanoparticles

This example illustrates the preparation and characterization of an alendronate-calcium carbonate nanoparticle suspension.

The alendronate-calcium carbonate nanoparticles were prepared by co-precipitating alendronate solution, calcium chloride and sodium bicarbonate. Briefly, 5 mL of alendronate (31.4 mg/mL in distilled water) was mixed with 1 mL of 0.5M Calcium Chloride (Sigma-Aldrich). The pH was measured to be 4.75. Slowly and with mixing, 6 mL of 0.1M Sodium Bicarbonate (Fluka). The solutions became cloudy as the particles precipitated. The final pH was 6.4.

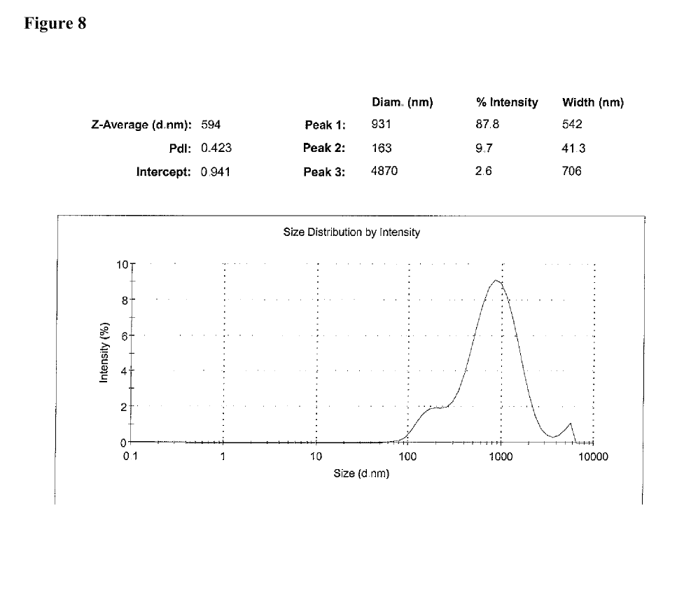

The particle size distribution of the alendronate-calcium carbonate nanoparticles is shown in FIG. 8.

In Vitro Cell Cytotoxicity Assay of Alendronate-Calcium Carbonate Nanoparticles.

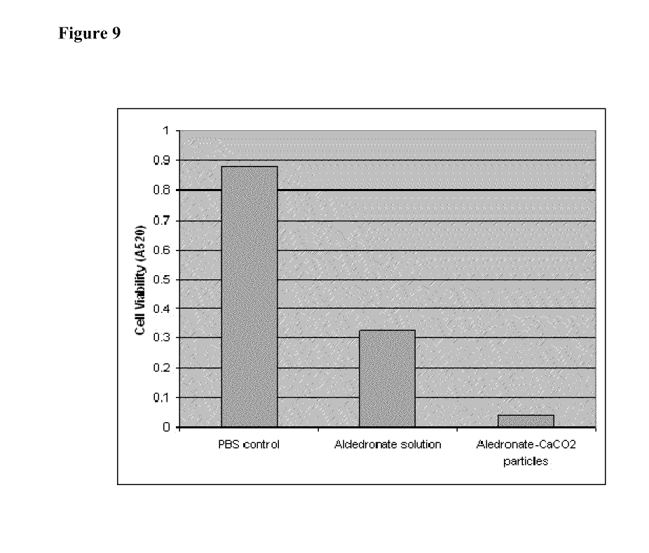

The effect of alendronate-calcium carbonate nanoparticles on the cell viability of the RAW 264.7 cell line was determined using the MTT assay. In brief, cells were plated into 96-well plates at 5% confluence and incubated with formulation at full strength (10% in DMEM culture medium) for 4 days. Media were removed and replaced with fresh RPMI medium containing MTT. Half of the cells were lysed before MTT addition as a control for background absorbance. The remaining unlysed cells were then lysed and OB 560 was determined. Five replicates were run for each condition. Results are depicted in FIG. 9. Alendronate-calcium carbonate nanoparticles significantly decreased the viability of RAW 264.7 cells relative to equal concentrations of alendronate in solution.

### Example 5

### Alendronate-PLG Nanospheres Formulation

This example illustrates PLG nanospheres of encapsulated alendronate formulation

The preparation of Alendronate-PLG nanospheres is carried out essentially as described previously (Epstein H. et al., The Open Card. Med. J. 2:60-69, 2008). Briefly, a modified double emulsion-solvent evaporation technique is used to prepare nanospheres of PLGA (poly(lactic-co-glycolic acid)) containing alendronate using 0.5 ml of polyvinyl alcohol, MW 30,000-70,000 (PVA, Sigma-Aldrich) as a 2.8% solution in Tris buffer pH 7.4 containing 20 mg alendronate is emulsified in 3 mL of dichloromethane containing 3% PLGA by sonification over an ice-bath using a probe-sonicator at 14 W output for 90 seconds. The resulting primary emulsion is added to a 2% PVA (20 mL) solution in Tris buffer pH 7.4 containing CaCl2 at a 2:1 molar ratio of calcium to alendronate and sonicated for 90 seconds at 18 W output over an ice bath to form a double emulsion. DCM is eliminated by 3 hour evaporation under magnetic stirring at 4° C.

The Alendronate-PLG nanospheres are characterized by size, distribution and alendronate loading. The mean diameter of the nanospheres will be determined to range from about 200 to 300 nanometers with an entrapment percentage of about 40-60%.

### Example 6

### Clodronate-BZT in PLG Nanospheres

The preparation of Clodronate—PLG nanospheres is carried out with modifications to that described in Example 5. Briefly, 100 mM clodronate is precipitated with 5 molar excess of BZT (benzethonium chloride, Sigma-Aldrich), centrifuged, rinsed with distilled water, dried and resuspended in dichloromethane. The Clodronate—PLG nanospheres are prepared using 0.5 mL of polyvinyl alcohol, MW 30,000-70,000 (PVA, Sigma-Aldrich) as a 2.8% solution in Tris buffer pH 7.4 emulsified with 3 mL of dichloromethane containing 3% PLGA (poly(lactic-co-glycolic acid)) and 20 mg clodronate-BZT by sonification over an ice-bath using a probe-sonicator. DCM is eliminated by 3 hour evaporation under magnetic stirring at 4° C.

The Clodronate-BZT-PLG nanospheres are characterized by size, distribution and clodronate loading.

What is claimed:

1. A method of treating or preventing the growth, invasion and/or metastasis of a tumor, comprising administering, to a subject having a tumor or at risk for developing a tumor, a composition comprising a bisphosphonate and a pharmaceutically acceptable carrier, the pharmaceutically acceptable carrier comprising non-liposomal particles.

2. The method of claim 1, wherein the non-liposomal particles are suitable for uptake by cancer associated myeloid derived cells.

3. The method of claim 2, wherein the non-liposomal particles are spheroid particles.

4. The method of claim 2, wherein the non-liposomal particles are non-spheroid particles.

5. The method of claim 2, wherein the non-liposomal particles have a mean diameter between about 10 nm and about 10,000 nm.

6. The method of claim 2, wherein the non-liposomal particles have a mean diameter between about 20 nm and about 1000 nm.

7. The method of claim 2, wherein the non-liposomal particles have a mean diameter between about 50 nm and about 500 nm.

8. The method of claim 5, wherein at least 90% of the non-liposomal particles have a diameter between about 20 nm and about 1000 nm.

9. The method of claim 1, wherein the free bisphosphonate compound is released from the composition upon uptake by a cancer associated myeloid derived cell.

10. The method of claim 9, wherein the bisphosphonate compound is non-covalently associated with the non-liposomal particles.

11. The method of claim 1, wherein the composition is administered directly to a tumor.

12. The method of claim 1, wherein the composition is administered intravenously.

13. The method of claim 1, wherein the composition is administered in an amount effective to reduce the number of cancer associated myeloid derived cells in the subject.

14. The method of claim 1, wherein the composition is administered in an amount effective to reduce the number of cancer associated myeloid derived cell progenitor cells in the subject.

15. The method of claim 1, wherein the composition has a lower affinity for bone than the free bisphosphonate compound.

16. The method of claim 1, wherein the non-liposomal particles do not bear a cancer targeting ligand.

17. A pharmaceutical composition for treating or preventing the growth, invasion and/or metastasis of a tumor, comprising a bisphosphonate and a pharmaceutically acceptable carrier, the pharmaceutically acceptable carrier comprising non-liposomal particles.

18. The pharmaceutical composition of claim 17, wherein the non-liposomal particles have a mean diameter between about 20 nm and about 1000 nm.

19. The pharmaceutical composition of claim 17, wherein the free bisphosphonate compound is released from the composition upon uptake by a cancer associated myeloid derived cell.

20. The pharmaceutical composition of claim 17, wherein the composition has a lower affinity for bone than the free bisphosphonate compound.
